(19) United States
(12) Patent Application Publication
(10) Pub. No.: US 2022/0378721 A1
(43) Pub. Date: Dec. 1, 2022

(54) PROTEIN KINASE INHIBITORS AND USE THEREOF FOR TREATMENT OF NEURODEGENERATIVE DISEASES

(71) Applicant: Xavier University of Louisiana, New Orleans, LA (US)

(72) Inventors: Jayalakshmi SRIDHAR, New Orleans, LA (US); Melyssa BRATTON, New Orleans, LA (US); Navneet GOYAL, New Orleans, LA (US); Vishwajeet JHA, Bihar (IN); Richard SCHROEDER, Boston, MA (US)

(21) Appl. No.: 17/771,391

(22) PCT Filed: Oct. 22, 2020
(86) PCT No.: PCT/US20/56797

§ 371 Date: Apr. 22, 2022

Related U.S. Application Data
(60) Provisional application No. 62/925,395, filed on Oct. 24, 2019.

(51) Int. Cl. A61K 31/122; A61P 25/28

(52) U.S. Cl. CPC A61K 31/122; A61P 25/28

(57) ABSTRACT

The present disclosure relates to compounds that act as protein kinase inhibitors, especially CK1δ and/or CK1ε inhibitors, which can be used to treat a serine threonine kinase-dependent disease and condition, such as neurodegenerative diseases like Alzheimer's Disease, and the synthesis of the same. Further, the present disclosure teaches the utilization of such compounds in a treatment for neurodegenerative diseases, including Alzheimer's disease.

### CROSS REFERENCE TO RELATED APPLICATIONS

This application claims priority to U.S. Provisional Patent Application No. 62/925,395, filed Oct. 24, 2019, the content of which is hereby incorporated by reference in its entirety.

### STATEMENT REGARDING FEDERALLY SPONSORED RESEARCH OR DEVELOPMENT

This invention was made with government support under Louisiana Biomedical Research Network, National Institute of Health Grant Number: 20GM103424. The government has certain rights in the invention.

The present disclosure relates to compounds that inhibit protein kinase, especially CK1δ and/or CK1ε, which can be used to treat a serine/threonine kinase-dependent disease and condition, such as neurodegenerative diseases like Alzheimer's Disease. The disclosure also relates to pharmaceutical compositions comprising these small molecule protein kinase inhibitors, and methods for using the same for treatment of serine threonine kinase-dependent diseases and conditions.

### BACKGROUND

### I. Field

The present disclosure relates to compounds having, for example, activities as protein kinase inhibitors, for example, CK1δ and/or CK1ε inhibitors, and methods for making the same. The disclosure also relates to pharmaceutical compositions comprising these protein kinase inhibitors, and methods for using the same for treatment of protein kinase-dependent diseases/conditions, including tauopathy evidenced in neurodegenerative diseases such as Alzheimer's disease, as well as other diseases in mammals.

The compounds described here can provide effective therapy for neurodegenerative diseases, such as Alzheimer's Disease.

### 2. Description of Related Art

Progressive neurodegenerative disorders that impair cognitive and behavioral symptoms, include Alzheimer's disease (AD) and several other dementias. In 2019, 5.8 million Americans are estimated to be living with AD with a worldwide estimate of 50 million people with AD in 201711-21. The projected cost estimate of AD in the US in 2019 is 290 billion and in 2050 is 1.1 trillion [1]. Without any progress in pharmacologic treatment for AD the worldwide number is expected to reach 152 million by 2050121. Currently there is no clinical therapeutic agent for the cure of AD and the only drugs approved by FDA are the cholinesterase inhibitors and memantine that can provide symptomatic treatment but do not alter the course of AD.

Two hallmarks of AD are the accumulation of β-amyloid (Aβ) plaques outside neurons and tau tangles (also called Neuro Fibrillary Tangles—NFTs) inside neurons [3-10]. Over the past decade growing research in AD has identified several key factors that play mechanistic role in the pathogenesis of AD including oxidative stress, mitochondrial function, alterations in neurotransmissions, changes in expression of several proteins affecting multiple molecular pathways [11-12]. Tauopathy, the aggregation of tau into neurofibrillary tangles (NFTs), is the primary pathological feature for more than 20 neurological disorders including Alzheimer's disease (AD), frontotemporal dementia with parkinsonism-17 (FTCP-17), progressive supranuclear palsy, and Parkinson's disease. Tau exists in an unfolded state and the majority (˜80%) interacts with microtubules in the axons of neurons. Therapeutic strategies pursued by researchers include targeting Af using monoclonal antibodies and secretase inhibitors, targeting tau using kinase inhibitors and tau aggregation inhibitors, targeting ApoE4 interaction with AP using small molecules, synaptic dysfunction [13-14], modulators of aging [15-16] or autophagy [17].

The target for the present therapeutics development is the targeting of the pathogenic event of hyperphosphorylated tau aggregation using kinase inhibitors.

In AD brain, tan is abnormally hyperphosphorylated causing disruption of microtubule through sequestration of normal tau, MAP1 and MAP2 leading to misfolding and co-aggregation into filaments [18-19]. It has been shown that abnormally hyperphosphorylated tau isolated from AD brain does not promote microtubule assembly or binding to microtubules and tau dephosphorylation restores biological activity of tau [20-23]. Many protein kinases are involved in tan phosphorylation including several members of the kinase groups AGC (Containing PKA, PKG, PKC families), CAMK (Calcium/calmodulin-dependent protein kinase), CK1 (Casein Kinase 1) and CMGC (Containing CDK, MAPK, GSK3, CLK families).

Among the 23 kinases that can phosphorylate the ˜85 possible sites in tau that were identified by immunohistochemical studies and mass spectral studies are the family of Casein kinase 1 (CK1) enzymes. The family of Casein kinase 1 enzymes is one of the most abundant among protein kinases to be found in the eukaryotic cells [24]. These are serine/threonine kinases that have multiple important roles to play in the regulation of DNA repair, circadian rhythm, meiotic progression, Wnt signaling, autophagy, ribosome assembly, tubule stabilization and intracellular trafficking [24-29]. The CK1 family is comprised of seven isozymes—α, β, γ1, γ2, γ3, δ and ε as well as splice variants of CK1α, δ, ε and γ3.

The isozymes could be grouped into three related ones based on their sequence alignment. CK1α and CK1β; CK1δ and CK1ε; and all of the three CK1γ isozymes (FIG. 1). The alignment score between CK1α and CK1β is 76.2 and between CK1δ and CK1ε is 81.9. CK1α has an identical alignment score of 67.7 with CK1δ and CK1ε. CK1β has a similar alignment score with CK1δ and CK1ε of 58.6 and 56.8, respectively. While the CK1 γ1, γ², γ3 isozymes are closely related to each other with an alignment score range of 77.7 to 79.5% between them, they are distant from the others by an alignment score of 41.2 to 48.1%. Among the seven isozymes of the CK1 family, CK1δ and CK1ε isozymes have the highest homology. The kinase domain of CK1δ and CK1ε are 98% identical while their C-terminal domain shows 53% identity leading to some redundancy in the substrate phosphorylation but with many distinct biological roles for these two isozymes of CK1. CK1δ is expressed in comparable levels in most human tissues while CK1ε is expressed in higher levels in the brain and endometrium. CK1δ and CK1ε are highly overexpressed in Alzheimer-affected brain and co-localize with neuritic and granulovacuolar lesions. Indeed, CK1δ and CK1ε protein expression is increased more than >30-fold and 9-fold, respectively, in the hippocampus of Alzheimer-affected brain compared with equivalent controls [49-50].

CK1α, CK1δ, and CK1ε have a common regulatory function [25, 29] and they act in a concerted way in the evolutionarily conserved Wnt/β-catenin signaling pathway (β-catenin, disheveled (DVL))[31], adenomatous polyposis coli (APC) [32], PI3K-AKT (Foxo1) [33], nuclear factor of activated T-cells, cytoplasmic 3 (NFATC3) [34], p53 (p53, MDM2) [35], and death receptor signaling (FADD) [36]. The Wnt/β-catenin signaling pathway is one of the few pathways that govern the equilibrium between proliferation and differentiation. CK1 isoforms are involved in other oncogenic signaling pathways such as regulation of cell cycle, apoptosis induction or cell survival. CX1α, CK1δ and CK1ε play important regulatory roles in the circadian rhythm of eukaryotic cells [37-39]. While all three of them are negative regulators of PER 1, CK1δ and ε seem to bind more strongly to PER1 than CK1α. The selective inhibition of CK1ε has minimal effect on the regulation of circadian rhythm revealing the redundancy of CK1ε when compared to pan CK1δ/ε inhibitors that prolonged the circadian rhythm [40]. CK1δ is known to regulate the phosphorylation of tubulins (α-, β- and γ-), microtubule associated proteins (MAPs), stathmin and tau at multiple sites (hereby playing a critical role in the stability and dynamics of microtubule and spindle apparatus [41-45]. Recent evidence has emerged for the role CK1ε in the phosphorylation of tau at several sites and suppressed tau exon 10 inclusion [46].

Casein kinase 1δ (CK1δ) and casein kinase 1ε (CK1ε) have been shown to phosphorylate tau at 36 and 7 sites, respectively, in in-vitro studies [47]. The binding of tau to the MT is regulated by the phosphorylation state of tau protein and experimental evidence points to Ser199, Ser202, Ser231, Thr205, Thr231, Ser262, Ser396 and Ser404 phosphorylation sites mediating this activity [47-48]. CK1δ/ε phosphorylates many of these sites in-situ resulting in the shift of tau-microtubule equilibrium towards free tau.

Recent reports have suggested that CK1 not only phosphorylates tau protein at several sites, it also provides priming activity for other kinases to hyperphosphorylate the tau protein. Overexpression of CK1δ increased tau phosphorylation at residues Ser202/Thr205 and Ser396/Ser404 in situ and decreased fraction of tau bound to microtubules. Their results lead to the conclusion that CK1δ phosphorylation sites on tau modulate tau/microtubule binding [48]. In addition to tau, other microtubule associated proteins MAP1A and MAP1B which are multimeric complexes consisting of heavy and light chains have been shown to be phosphorylated by CK1δ. Two domains on light chain LC2 have been found to be phosphorylated by CK1δ which could lead to alteration of microtubule dynamics [51].

Over the last decade several researchers have identified and developed small molecule CK1 inhibitors and some of them have an inhibition profile that is isoform specific. These small molecules have belonged to pyrimide [52], imidazole [53], benzimidazole [54], phenyl-indazole [55], indole [56], and the aminoanthraquinone [57] classes of molecular scaffolds.

However, selective inhibitors of CK1ε and/or CX1δ can function as in-vivo tools to decipher the distinct roles of these isozymes in diseases such as cancer and neurodegenerative disorders. Therefore, there is a market need for new class of compounds as selective CK1δ and/or CK1ε inhibitors which can be used as pharmaceuticals for neurodegenerative diseases such as Alzheimer's disease, among other diseases. Targeting protein kinases, such as CK1δ and/or CK1ε, using small molecule protein kinase inhibitors would be a very effective strategy for treating Alzheimer's disease as serine/threonine kinase inhibitors may be more effective in inhibiting tau hyperphosphorylation at specific residues associated with microtubule binding.

### BRIEF SUMMARY

The present disclosure relates generally to compounds and compositions useful for the inhibition of serine/threonine kinases, such as CK1δ and/or CK1ε; compounds, intermediates, and methods of making such compounds and compositions; methods of using such compounds and compositions; pharmaceutical compositions comprising such compounds and compositions; and methods of using such pharmaceutical compositions.

In an embodiment, the present serine/threonine kinase inhibitors are CK1δ and/or CK1ε inhibitors.

In an embodiment, the present invention provides derivatives of emodin (formula (I)), or a stereoisomer or pharmaceutically acceptable salt thereof.

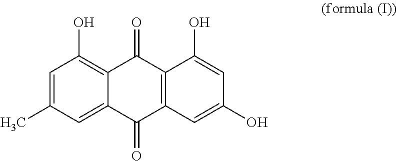 [SMILES: Cc1cc(O)c2c(c1)C(=O)c1cc(O)cc(O)c1C2=O]

In an embodiment, the present invention provides a compound of formula (II) or a stereoisomer or pharmaceutically acceptable salt thereof.

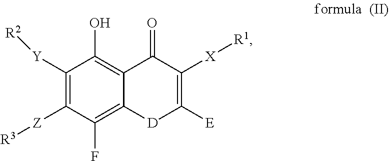

wherein:
X, Y and Z independently represent a direct bond, —C(R)—, —O—, —S—, —OH, —NH₂, —CH₂O—, CH₂S—, —(CH₂)₂O—, —NR⁵—, —NR⁵CH₂—, —CH₂NR⁵—, —NR⁵CO—, —CONR⁵—, —N═N—, —NH—CO—NH—, —NH—CS—NH—, —CO—O—, CO—O—CH₂—, —SO₂NH—, —NH—SO₂—, —CR⁴═CR⁴—, —C≡C—, —O—CH₂—CO—, —OCH₂CHO—, —CH(OH)—, —NO₂ bridging groups,
R⁴ represents hydrogen, C₁₋₆ alkyl, C₁₋₆ alkenyl, C₁₋₆ alkoxy, C₁₋₆haloalkyl, haloC₁₋₆ alkoxy, —OH, —(═O), —COOH, —CONH₂, —COC₁₋₆alkyl, O—C₁₋₆ alkyl or alkenyl or alkynyl, NH—C₁₋₆ alkyl or alkenyl or alkynyl, —SC₁₋₆ alkyl groups, —(═S), CSSH, CSNH₂, —CSC₁₋₆ alkyl or alkenyl or alkynyl, S—C₁₋₆ alkyl or alkenyl or alkynyl
R¹, R², R³ and R⁵ independently represent hydrogen, C₁₋₆ alkyl, alkenyl, alkynyl, halogenated or hydroxyl alkyl, alkenyl, alkynyl, halogenated or hydroxyl or amino-alkenyl, halogenated or hydroxyl or amino-alkynyl, halogen, aryl, C₃₋₈ cycloalkyl, monocyclic or bicyclic heterocyclyl, monocyclic or bicyclic heteroaryl, wherein the aryl, heteroaryl or heterocyclyl groups may be optionally substituted by one or more R⁴ groups.
D represents —(C═O)—, —(CH₂)ₙ— where n=0, 1, 2, —CHOH—, CHNH₂—, —O—, —S—, —NH—, —N—CH₃—,
E represents hydrogen, C₁₋₆ alkyl, halogen, —OH, aryl, halogenated-hydroxyl aryl, heteroaryl, halogenated or hydroxyl or amino-heterocyclyl, cycloalkyl, halogenated or hydroxyl alkyl, alkenyl, alkynyl, halogenated or hydroxyl or amino-alkenyl, halogenated or hydroxyl or amino-alkynyl,
F represents hydrogen, C₁₋₆ alkyl, —OH, —NH2, NHCOCH₃, NHCOR¹, aryl, halogenated/hydroxyl aryl, heteroaryl, halogenated or hydroxyl or amino-heterocyclyl, cycloalkyl, halogenated or hydroxyl alkyl, alkenyl, alkynyl, halogenated or hydroxyl or amino-alkenyl, halogenated or hydroxyl or amino-alkynyl;
wherein the compound is useful for inhibition of serine/threonine kinases, such as CK1δ and/or CK1ε.

In a further embodiment,

X, Y and Z independently represent a direct bond, —C(R⁴)—, or —CH(OH)—,
R⁴ represents hydrogen, C₁₋₆ alkyl, halogen, —(═C) or —OH,

R¹, R², and R³ independently represent hydrogen, C₁₋₆ alkyl, halogen, aryl, C₃₋₈ cycloalkyl, monocyclic or bicyclic heterocyclyl, monocyclic or bicyclic heteroaryl, wherein the aryl, heteroaryl or heterocyclyl groups may be optionally substituted by one or more R⁴ groups,

D represents —(C═O)—,
E represents aryl, hydrogen, C₁₋₆ alkyl, or halogen,
F represents hydrogen, C₁₋₆ alkyl, or —OH.

In a further embodiment, D is —(C═O)—, and F is —OH or H.

In a further embodiment, D is —(C═O)—, and F is —OH.

In a further embodiment, the halogen is Cl or Br.

In a further embodiment, the compound of formula (II) is an inhibitor of CK1δ, and X, Y and Z independently represent a direct bond, —C(R⁴)—, or —CH(OH)—,

R⁴ represents halogen,
R¹, R², and R³ independently represents hydrogen, halogen, or C₁₋₆ alkyl,
D represents —(C═O)—.
E represents hydrogen, and
F represents —OH.

In a further embodiment, the compound of formula (II) is an inhibitor of CK1ε, and X, Y and Z independently represent a direct bond,

R¹, R², and R³ independently represent hydrogen or halogen,
D represents —(C═O)—,
E represents hydrogen or halogen,
F represents —OH, and
the compound is an inhibitor of CK1ε.

In a further embodiment, the compound of formula (II) is an inhibitor of CK1ε, and X, Y and Z independently represent a direct bond or —C(R⁴)—

R⁴ represents hydrogen, C₁₋₆ alkyl,
R¹, R², and R³ independently represent hydrogen, C₁₋₆ alkyl, aryl, C₃₋₈ cycloalkyl, monocyclic or bicyclic heterocyclyl, monocyclic or bicyclic heteroaryl, wherein the aryl, heteroaryl or heterocyclyl groups may be optionally substituted by one or more R⁴ groups,
D represents —(C═O)—,
E represents hydrogen or halogen,
F represents hydrogen.

The following compounds are surprisingly found to be CK1δ inhibitors, which can and subsequently also inhibit tau phosphorylation and be effective therapeutic agents for neurodegenerative diseases, such as Alzheimer's disease:

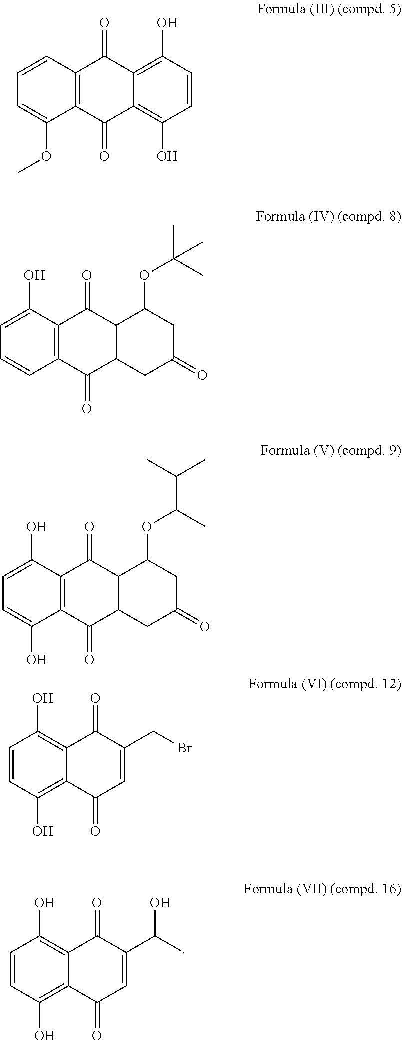

The following compounds are surprisingly found to be CK1ε inhibitors, which can and subsequently also inhibit tau phosphorylation and be effective therapeutic agents for neurodegenerative diseases, such as Alzheimer's disease:

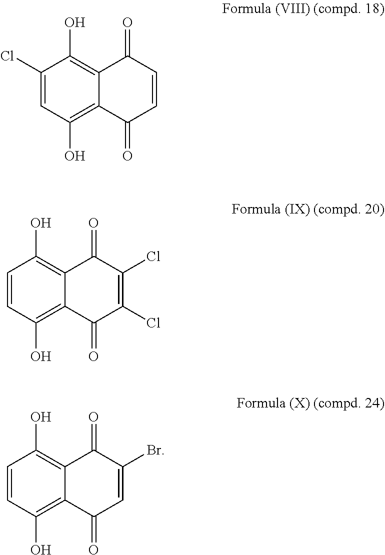 [SMILES: O=C1C=CC(=O)c2c(O)c(Cl)cc(O)c21 ; O=C1C(Cl)=C(Cl)C(=O)c2c(O)ccc(O)c21 ; O=C1C=C(Br)C(=O)c2c(O)ccc(O)c21]

In an embodiment, there is provided a pharmaceutical composition comprising at least one compound of formula (I) or a pharmaceutically acceptable salt or solvate thereof. In an embodiment, the pharmaceutical compound is for use in treating a patient who has, or in preventing a patient from getting, a disease such as neurogenerative disease. The compounds, compositions, and methods of the invention are useful for treating humans who have Alzheimer's Disease (AD), for helping prevent or delay the onset of AD, for treating patients with mild cognitive impairment (MCI), and/or preventing or delaying the onset of AD in those patients who would otherwise be expected to progress from MCI to AD. A further embodiment may provide a method of treating the neurodegenerative disease comprising administering to a subject a compound according to any one of the preceding paragraphs. An embodiment may provide use of a compound as in the paragraphs above for treating Alzheimer's disease. In some embodiments a compound as presented above is used in the preparation of a medicament for treatment of Alzheimer's disease.

The pharmaceutical compositions of the present disclosure can be in any form known to those of skill in the art. For instance, in some embodiments the pharmaceutical compositions are in a form of a product for oral delivery, said product form being selected from a concentrate, dried powder, liquid, capsule, pellet, and pill. In other embodiments, the pharmaceutical compositions are in the form of a product for parenteral administration including intravenous, intradermal, intramuscular, and subcutaneous administration. The pharmaceutical compositions may also further comprise carriers, binders, diluents, and excipients.

Also, in other aspects, the present disclosure relates to a serine/threonine kinase inhibitor composition comprising one or more compounds selected from the compounds of Formula (I) and (II), and pharmaceutically acceptable salts and solvates thereof. In an embodiment, said compound has a purity of ≥75%, ≥80%, ≥85%, ≥90%, ≥95%, ≥96%, ≥97%, or ≥98%, and ≥99%. In an embodiment, a pharmaceutical composition is provided comprising the claimed serine/threonine kinase inhibitor composition, either alone or in combination with at least one additional therapeutic agent, with a pharmaceutically acceptable carrier; and uses of the claimed serine/threonine kinase inhibitor compositions, either alone or in combination with at least one additional therapeutic agent, in the treatment of neurodegenerative diseases including Alzheimer's disease at any stage of the disease diagnosis. The combination with an additional therapeutic agent may take the form of combining the claimed serine/threonine kinase inhibitor compounds with any known therapeutic agent.

The methods for treating a clinical indication by the serine/threonine kinase inhibitor compounds disclosed herein, may be effectuated by administering a therapeutically effective amount of the serine/threonine kinase inhibitor compounds to a patient in need thereof, this therapeutically effective amount may comprise administration of the prodrug to the patient at 1 mg/kg/day, 2 mg/kg/day, 3 mg/kg/day, 4 mg/kg/day, 5 mg/kg/day, 10 mg/kg/day and 20 mg/kg/day. Alternatively, amounts ranging from about 0.001 mg/kg/day to about 0.01 mg/kg/day, or about 0.01 mg/kg/day to about 0.1 mg/kg/day, or about 0.1 mg/kg/day to about 1 mg/kg/day, or about 1 mg/kg/day to 10 mg/kg/day, or about 10 mg/kg/day to about 25 mg/kg/day are also contemplated.

A further object of the disclosure is a kit, comprising a composition containing at least one serine/threonine kinase inhibitor compounds disclosed herein for treatment and prevention of neurodegenerative diseases and related morbidities. The composition of the kit may comprise at least one carrier, at least one binder, at least one diluent, at least one excipient, at least one other therapeutic agent, or mixtures thereof.

One aspect of the present disclosure is the compounds disclosed herein as well as the intermediates as used for their synthesis.

While certain features of this invention shown and described below are pointed out in the annexed claims, the invention is not intended to be limited to the details specified, since a person of ordinary skill in the relevant art will understand that various omissions, modifications, substitutions, and changes in the forms and details of the invention illustrated and in its operation may be made without departing in any way from the spirit of the present invention. No feature of the invention is critical or essential unless it is expressly stated as being “critical” or “essential.”

These and other features, aspects, and advantages of embodiments of the present disclosure will become better understood with regard to the following descriptions, claims, and accompanying drawings explained below.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

For a further understanding of the nature, objects, and advantages of the present disclosure, reference should be had to the following detailed description, read in conjunction with the following drawings, wherein like reference numerals denote like elements.

FIG. 1 shows structures of Emodin, a known compound, and compounds 2-16 (compounds 2-11 are purchased and compounds 12-16 are synthesized) investigated for inhibition of CK1δ.

FIG. 2 shows docking studies of Emodin and compounds 3 and 5 from FIG. 1 in the ATP binding site of X-ray crystal structure of CK1δ. (A), (B) and (C) depict the binding modes of emodin, compounds 3 and 5, respectively. The ribbon model of the protein is shown. The protein residues are shown as stick models with the carbons in gray color; the ligand molecules are shown as ball and stick models. (D), (E) and (F) depict the various ligand interactions with the protein residues for emodin, compounds 3 and 5, respectively.

FIG. 3A and FIG. 3B show inhibition of Tau phosphorylation by CK1δ inhibitors in HeLa cells. HeLa cervical cancer cells ectopically expressing full-length Tau protein were treated with 10 μM of the indicated compounds. Samples were prepared in triplicate. FIG. 3A shows total Tau, pTau (serine 202), and actin levels, which were detected using capillary electrophoresis and the appropriate antibodies. Actin was used as a loading control only and was not used in any later calculations. FIG. 3B shows quantification of the pTau and Tau bands using the Compass software (Protein Simple) to determine areas under the curve of each band. The Y axis reflects the average change in pTau levels divided by total Tau levels for the triplicate experiments for each compound. For normalization purposes, the vehicle control was set to 1.0 and all other values were adjusted accordingly.

FIG. 4A shows sequence alignment tree showing the distance and relationships between the isozymes of CK1 family. FIG. 4B shows alignment score between the CK1 isozymes. FIG. 4C shows structure of CK1ε selective inhibitor PF-4800567.

FIG. 5 shows structures of some naphthoquinone compounds synthesized (compounds 25-30 are new).

FIG. 6 shows schemes 1 through 3 for synthesis of some of the new compounds.

FIG. 7 shows docking of compound 10 in the ATP binding pocket of CK1δ (green) and CK1ε (pink). FIG. 7 panel (A) shows the structure of compound 10. FIG. 7 panel (B) shows the molecular surface of ATP-binding pocket of CK1δ and CK1ε superposed, molecular surface colored by lipophilicity (green) and hydrophobicity (pink). FIG. 7 panel (C) shows binding mode of compound 10 with CK1δ and CK1ε, the hydrogen bond interactions are shown as red broken lines and the Phe150-bromine interactions are shown with distances as green broken lines. FIG. 7 panel (D) shows orientation of Phe150 in CK1δ and CK1ε, and the flip of the DDG motif in CK1ε is shown in comparison to CK1δ.

### DETAILED DESCRIPTION

Before the subject disclosure is further described, it is to be understood that the disclosure is not limited to the particular embodiments of the disclosure described below, as variations of the particular embodiments may be made and still fall within the scope of the appended claims. It is also to be understood that the terminology employed is for the purpose of describing particular embodiments, and is not intended to be limiting. Instead, the scope of the present disclosure will be established by the appended claims.

In this specification and the appended claims, the singular forms “a,” “an,” and “the” include plural reference unless the context clearly dictates otherwise. Unless defined otherwise, all technical and scientific terms used herein have the same meaning as commonly understood to one of ordinary skill in the art to which this disclosure belongs.

As used herein, the term “minimize” or “reduce”, or derivatives thereof, include a complete or partial inhibition of a specified biological effect (which is apparent from the context in which the terms “minimize” or “reduce” are used).

As used herein, the term “neurodegenerative diseases” refer to diseases which cause disruption to neurological function as the disease progresses. Common examples of neurodegenerative diseases include diseases such as Alzheimer's disease, multiple sclerosis, amyotrophic lateral sclerosis, dementia, Parkinson's disease, and Huntington's disease.

The compounds according to the disclosure are isolated and purified in a manner known per se, e.g. by distilling off the solvent in vacuo and recrystallizing the residue obtained from a suitable solvent or subjecting it to one of the customary purification methods, such as chromatography on a suitable support material. Furthermore, reverse phase preparative HPLC of compounds of the present disclosure which possess a sufficiently basic or acidic functionality, may result in the formation of a salt, such as, in the case of a compound of the present disclosure which is sufficiently basic, a trifluoroacetate or formate salt for example, or, in the case of a compound of the present disclosure which is sufficiently acidic, an ammonium salt for example. Salts of this type can either be transformed into its free base or free acid form, respectively, by various methods known to the person skilled in the art, or be used as salts in subsequent biological assays. Additionally, the drying process during the isolation of compounds of the present disclosure may not fully remove traces of cosolvents, especially such as formic acid or trifluoroacetic acid, to give solvates or inclusion complexes. The person skilled in the art will recognize which solvates or inclusion complexes are acceptable to be used in subsequent biological assays. It is to be understood that the specific form (e.g., salt, free base, solvate, inclusion complex) of a compound of the present disclosure as isolated as described herein is not necessarily the only form in which said compound can be applied to a biological assay in order to quantify the specific biological activity.

One aspect of the disclosure is salts of the compounds according to the disclosure including all inorganic and organic salts, especially all pharmaceutically acceptable inorganic and organic salts, particularly all pharmaceutically acceptable inorganic and organic salts customarily used in pharmacy.

Examples of salts include, but are not limited to, lithium, sodium, potassium, calcium, aluminum, magnesium, titanium, meglumine, ammonium, salts optionally derived from NH₃ or organic amines having from 1 to 16 C-atoms such as, e.g., ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine, N-methylpiperidine and guanidinium salts.

The salts include water-insoluble and, particularly, water-soluble salts.

As used herein, “pharmaceutically acceptable salts” refer to derivatives of the compounds disclosed herein wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. In the salt form, it is understood that the ratio of the compound to the cation or anion of the salt may be 1:1, or any ratio other than 1:1, e.g., 3:1, 2:1, 1:2, or 1:3.

It should be understood that all references to pharmaceutically acceptable salts include solvent addition forms (solvates) or crystal forms (polymorphs) as defined herein, of the same salt.

Salts of the compounds of formulas (I)-(X) according to the disclosure can be obtained by dissolving the free compound in a suitable solvent (for example a ketone such as acetone, methylethylketone or methylisobutylketone, an ether such as diethyl ether, tetrahydrofuran or dioxane, a chlorinated hydrocarbon such as methylene chloride or chloroform, or a low molecular weight aliphatic alcohol such as methanol, ethanol or isopropanol) which contains the desired acid or base, or to which the desired acid or base is then added. The acid or base can be employed in salt preparation, depending on whether a mono- or polybasic acid or base is concerned and depending on which salt is desired, in an equimolar quantitative ratio or one differing therefrom. The salts are obtained by filtering, reprecipitating, precipitating with a non-solvent for the salt or by evaporating the solvent. Salts obtained can be converted into the free compounds which, in turn, can be converted into salts. In this manner, pharmaceutically unacceptable salts, which can be obtained, for example, as process products in the manufacturing on an industrial scale, can be converted into pharmaceutically acceptable salts by processes known to the person skilled in the art.

According to the person skilled in the art the compounds of formulas (I) through (X) according to this disclosure as well as their salts may contain, e.g., when isolated in crystalline form, varying amounts of solvents. Included within the scope of the disclosure are therefore all solvates and in particular all hydrates of the compounds of formulas (I) through (II) according to this disclosure as well as all solvates and in particular all hydrates of the salts of the compounds of formulas (I) through (II) according to this disclosure.

“Solvate” means solvent addition forms that contain either stoichiometric or non stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

The compounds according to the disclosure and their salts can exist in the form of tautomers which are included in the embodiments of the disclosure.

“Tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerization is possible, a chemical equilibrium of the tautomers will be reached. The exact ratio of the tautomers depends on several factors, including temperature, solvent and pH. The concept of tautomers that are interconvertible by tautomerizations is called tautomerism.

Where the present specification depicts a compound prone to tautomerization, but only depicts one of the tautomers, it is understood that all tautomers are included as part of the meaning of the chemical depicted. It is to be understood that the compounds disclosed herein may be depicted as different tautomers. It should also be understood that when compounds have tautomeric forms, all tautomeric forms are intended to be included, and the naming of the compounds does not exclude any tautomer form.

Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

Common tautomeric pairs are: ketone-enol, amide-nitrile, lactam-lactim, amide-imidic acid tautomerism in heterocyclic rings (e.g., in nucleobases such as guanine, thymine and cytosine), imine-enamine and enamine-enamine.

The compounds of the disclosure may, depending on their structure, exist in different stereoisomeric forms. These forms include configurational isomers or optically conformational isomers (enantiomers and/or diastereoisomers including those of atropisomers). The present disclosure therefore includes enantiomers, diastereoisomers as well as mixtures thereof. From those mixtures of enantiomers and/or diastereoisomers pure stereoisomeric forms can be isolated with methods known in the art, preferably methods of chromatography, especially high performance liquid chromatography (HPLC) using achiral or chiral phase. The disclosure further includes all mixtures of the stereoisomers mentioned above independent of the ratio, including the racemates.

The compounds of the disclosure may, depending on their structure, exist in various stable isotopic forms. These forms include those in which one or more hydrogen atoms have been replaced with deuterium atoms, those in which one or more nitrogen atoms have been replaced with ¹⁵N atoms, or those in which one or more atoms of carbon, fluorine, chlorine, bromine, sulfur, or oxygen have been replaced by the stable isotope of the respective, original atoms.

Some of the compounds and salts according to the disclosure may exist in different crystalline forms (polymorphs) which are within the scope of the disclosure.

It is a further object of the disclosure to provide serine/threonine kinase inhibitor compounds disclosed herein, methods of synthesizing the serine/threonine kinase inhibitor compounds, methods of manufacturing the serine/threonine kinase inhibitor compounds, and methods of using the serine/threonine kinase inhibitor compounds. The compounds can also be made by synthetic schemes well established in the art. In an embodiment, the present serine/threonine kinase inhibitors are selective CK1δ and/or CK1ε inhibitors.

Another object of the disclosure is to provide a composition, for example a pharmaceutical composition, comprising at least one serine/threonine kinase inhibitor compound disclosed herein in an amount effective for the indication of diseases. In an embodiment, the disease is a neurodegenerative disease. In an embodiment, the disease is Alzheimer's disease.

In an embodiment, the object of such treatment is to inhibit serine/threonine kinases. In an embodiment, the serine/threonine kinases to be inhibited by the serine/threonine kinase inhibitor compounds disclosed herein are CK1δ and/or CK1ε.

As used herein, “treating” means administering to a subject a pharmaceutical composition to ameliorate, reduce or lessen the symptoms of a disease. As used herein, “treating” or “treat” describes the management and care of a subject for the purpose of combating a disease, condition, or disorder and includes the administration of a compound disclosed herein, or a pharmaceutically acceptable salt, polymorph or solvate thereof, to alleviate the symptoms or complications of a disease, condition or disorder, or to eliminate the disease, condition or disorder. The term “treat” may also include treatment of a cell in vitro or an animal model. As used herein. “subject” or “subjects” refers to any animal, such as mammals including rodents (e.g., mice or rats), dogs, primates, lemurs or humans.

Treating the neurogenerative disease such as Alzheimer's disease may result in preventing or delaying or halting the progression of Alzheimer's disease.

### EXAMPLES

Hereby are provided non-limiting examples of embodiments of compounds disclosed herein.

### Example 1: CK1δ Inhibitors

Emodin, an ingredient of Chinese herbal medicines, which is a known inhibitor of several kinases, was the starting point in the search for new class of kinase inhibitors. Similarity search using Sybyl-UNIT Y search was performed of the PUBCHEM and ZINC databases. Over 40 structurally similar compounds were purchased and analyzed for their inhibition of key disease relevant kinases.

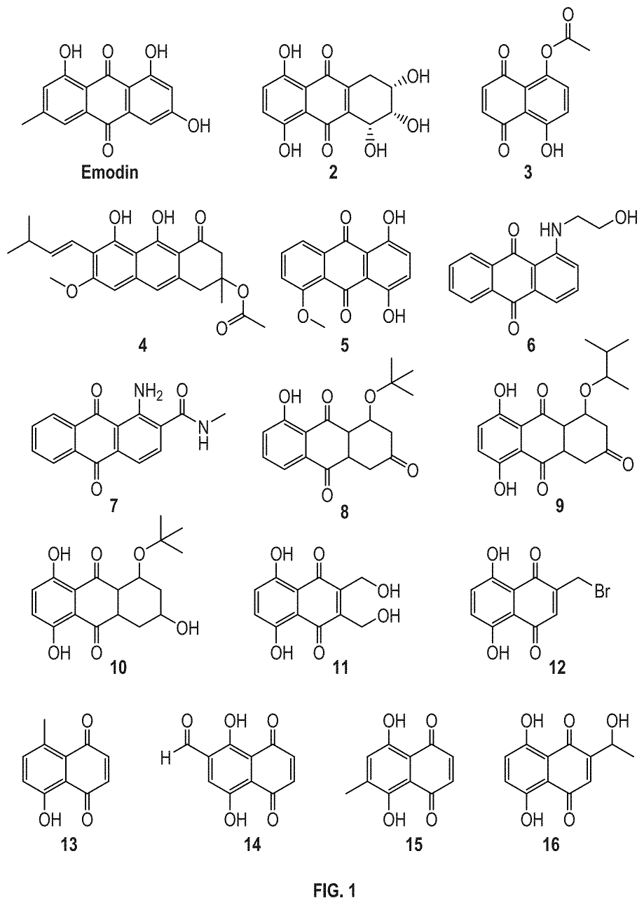

Compound 2 (from FIG. 1) was screened against a panel of 100 kinases to understand the specificity. It was found to strongly inhibit three kinases at 10 μM concentration-CK1δ (70%), Pim1 (66%) and Pim3 (64%) in a selective manner over 97 other kinases (Table 1).

TABLE 1
Kinases that were inhibited at >60%
by compound 2 in a100 kinase panel assay.

| | % Inhibition |
|---|---|
| Kinase | at 10 μM |
| CSNK1D | 70 |
| CSNK1G2 | 0 |
| PIM1 | 66 |
| PIM3 | 64 |

The inhibition of CK1δ (70%) by compound 2 was more selective over its isozyme CK1γ2 (no inhibition). Based on these results, the search began to understand the structural features of this class of compounds that can impart a higher potency of inhibition with similar selectivity profile for CK1δ. Using structure similarity search feature UNITY in SYBYL, 28 compounds with similar structures was identified, the compounds purchased from Specs Chemical Repository and analyzed for their ability to inhibit CK1δ.

Casein kinase 1δ Inhibition Assay: An initial high-throughput assay of the 28 commercially purchased compounds and 22 synthesized compounds were conducted at a 10 μM concentration at the ThermoFisher Select Screen facility. 9 of the commercial compounds and 5 of the synthesized compounds showed >80% inhibition of CK1δ in the FRET based assays. FIG. 1 sets forth their chemical structures.

These compounds were then subjected to dose response curve determination studies with the same FRET based assays at ThermoFisher Scientific. The IC₅₀ values, defined as the concentration of the compound required to inhibit cell proliferation by 50%, of the some of the compounds from the screening were measured (Table 2).

TABLE 2
IC₅₀ values of inhibition of CK1δ by compounds 3 to 16

| | CK1δ |
|---|---|
| Compound | (IC₅₀ μM) |
| 3 | 0.2 |
| 4 | 16.1 |
| 5 | 0.2 |
| 6 | 21.2 |
| 7 | 19.4 |
| 8 | 18.9 |
| 9 | 5.8 |
| 10 | 10.1 |
| 11 | 27.2 |
| 12 | 0.7 |
| 13 | 31.2 |
| 14 | 3.1 |
| 15 | 6.3 |
| 16 | 2.4 |

As seen in Table 2 above, the compounds showed inhibition of CK1δ with IC₅₀ values varying from 0.2 to 27.2 μM. Compound 3, 5 and 12 showed submicromolar IC₅₀ values of CK1δ inhibition of 0.2, 0.2, and 0.7 μM. Compound 13 exhibited the least inhibition potency with an IC₅₀ value of 31.2 μM (Table 2).

Synthetic strategies to derive the derivatives: Three different synthetic strategies were adopted for the overall synthesis of the derivatives with varying side chains and functionalities. The first synthetic scheme started with the functionalization of tetramethoxynaphthalene using a known organic reaction-formylation followed by oxidation of one of the rings to the quinone moiety using ceric ammonium nitrate oxidation. The second synthetic strategy used the Friedel Crafts double acylation of a substituted benzene ring with suitably substituted maleic anhydrides in the presence of aluminum chloride and sodium chloride. The third synthetic strategy made use of Diels Alder [4+2] cycloaddition reaction using 3-methyl-1-methoxy-1-trimethylsiloxy-1,4-diene and 1,4-benzoquinone as a dienophile. The synthesized compounds were also analyzed for their ability to inhibit CK1δ and five compounds (compounds 12 to 16, FIG. 1) from this series were found be effective in CK1δ inhibition (Table 2).

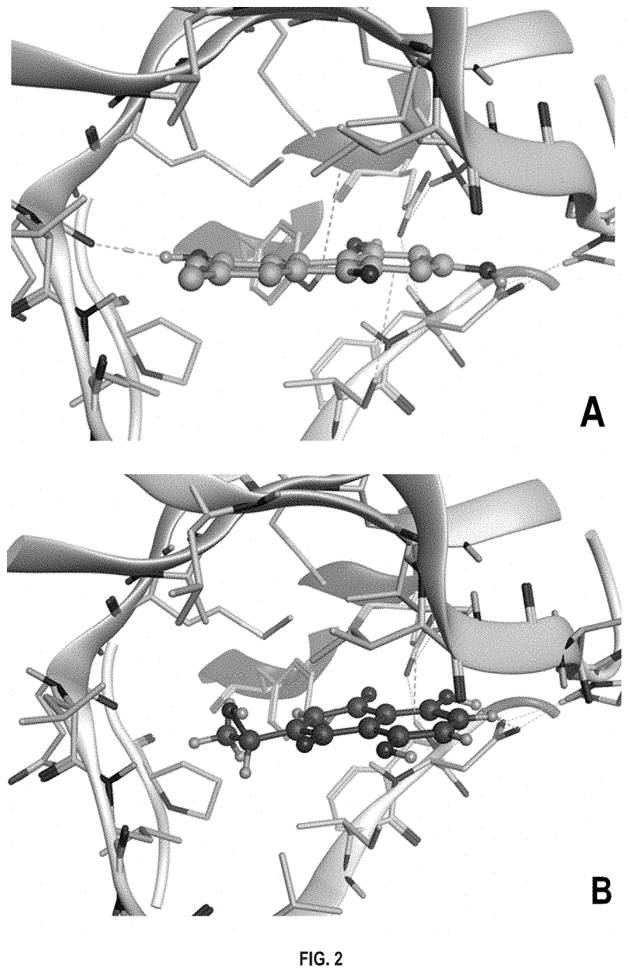

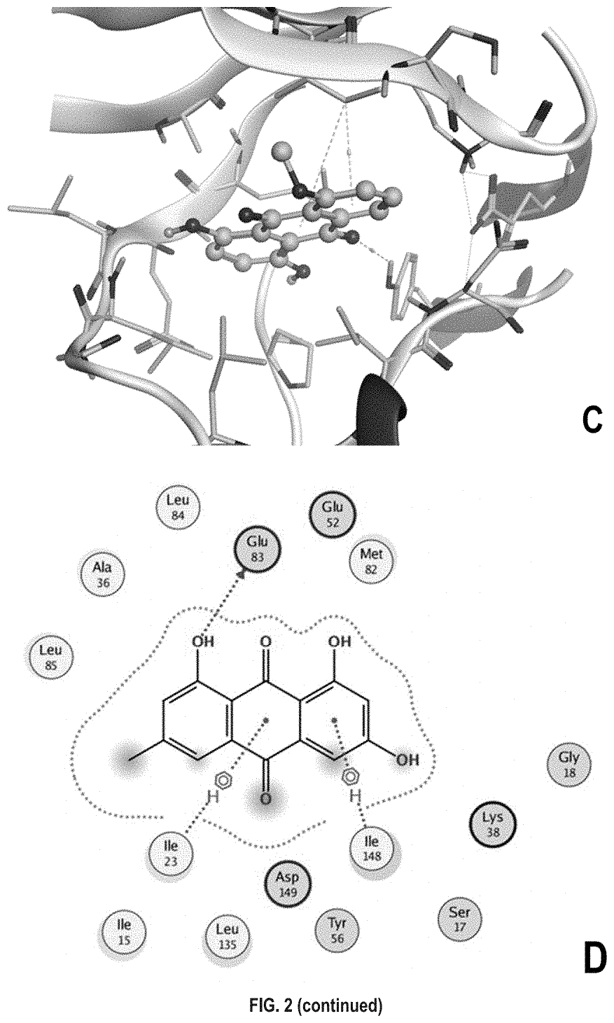

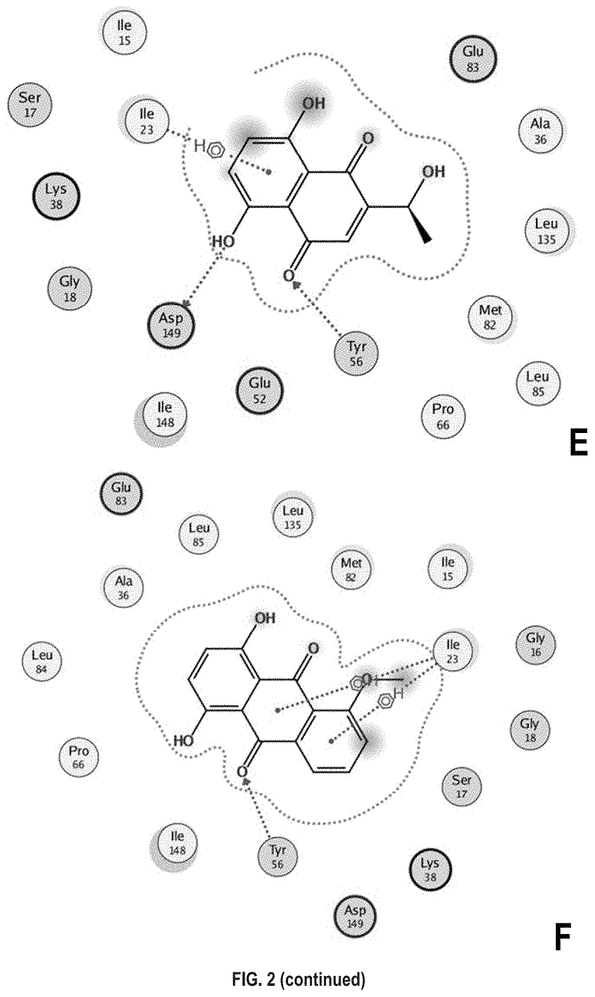

Docking Studies: Docking studies as set forth in FIG. 2 reveal the binding postures of the CK1δ inhibitors and the residues that are largely targeted by some of these compounds. Emodin and compounds 2 to 16 were docked onto the X-Ray crystal structure of CK1δ. FIG. 2 shows the docking studies for Emodin and compounds 3 and 5. Emodin formed a hydrogen bond with the hinge region residue Glu83. Aromatic rings of the molecules also made π-methyl interactions with Ile23 and Ile148. Many of the compounds adopted a conformational posture different form that of emodin in the binding pocket of CK1δ. The carbonyls of the quinone moieties formed a hydrogen bond with the phenolic hydroxyl group of Tyr56. The aromatic π-methyl interaction with Ile23 was exhibited by all of the compounds. Compound 3 made two hydrogen bonds-, the quinone carbonyl made a hydrogen bond with Tyr56 and the side chain carbonyl of compound 3 formed a hydrogen bond with the backbone amine group of the hinge region residue Leu85. Compound 5 showed a similar hydrogen bond of the quinone carbonyl with Tyr56, but did not form any hydrogen bonds with the hinge region. Compound 12 shifted more towards the triad residues Asp-Phe-Gly on the catalytic loop with the quinone carbonyl forming the hydrogen bond with Tyr56 and a second hydrogen bond by the phenolic hydroxyl group with Asp149.

In the ATP-binding pocket of the kinase, the investigated compounds were positioned closer to the triad residues Asp-Phe-Gly in the catalytic loop than the hinge region. Some compounds with three rings and two-ring compounds with suitable side chains were able to span the width of the pocket and reach out to the hinge region residues for hydrogen bonding. For example, compound 3 with the side chain carbonyl makes a hydrogen bond with hinge residue Leu85. One of the common features found for all of the compounds was that the phenolic hydroxyl group of the Tyr56 residue on the C-helix is forming a hydrogen bond with the carbonyl of the quinone ring of the present series of compounds. Depending on the orientation of the compound in the binding pocket, the hydroxyl group that is ortho to the quinone carbonyl also forms a hydrogen bond with the triad Asp 149 side chain carboxyl group. Additionally, all of the compounds exhibited aromatic π-methyl interactions with the residue Ile23. Compounds 8, 9 and 10 exhibited 1 to 2 hydrogen bonds with the hinge region residues, but the large hydrophobic branched alkyl groups were in general pushed to the periphery of the binding pocket with the large alkyl groups oriented outwards and exposed to solvent. The alkyl side chains have close proximity to the nonpolar residues Ile148 and Leu138 with high probability for hydrophobic interactions with their side chains. However, the exposure of these alkyl chains to solvent can contribute to unfavorable environment which could lead to these compound's relatively lower IC₅₀ values.

For a CK1δ inhibitor to be considered a potential therapeutic for AD, the compound should be able to inhibit the phosphorylation of tau at the specific residues that are known to be phosphorylated by CK1δ. It has been shown that tau phosphorylation at Ser202/Ser205 and Ser396/Ser404 by CK1δ is not dependent on the priming by other protein kinases. Additionally, these phosphorylation sites are among those sites (Ser199, Ser 202, Ser231, Thr205, Ser396 and Ser404) that regulate the microtubule stabilizing function. Based on these evidences, Ser202 was chosen as the representative tan phosphorylation site of this study to understand the efficacy of our CK1δ inhibitors in inhibiting tau phosphorylation.

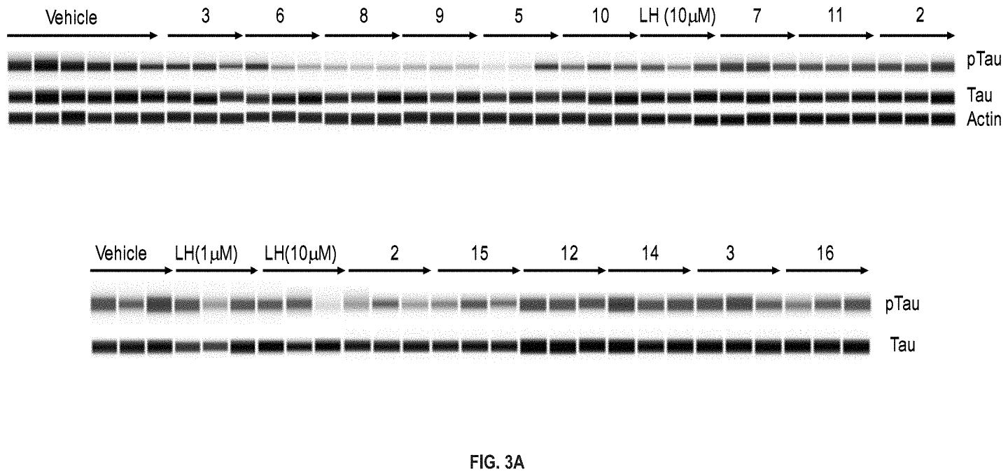

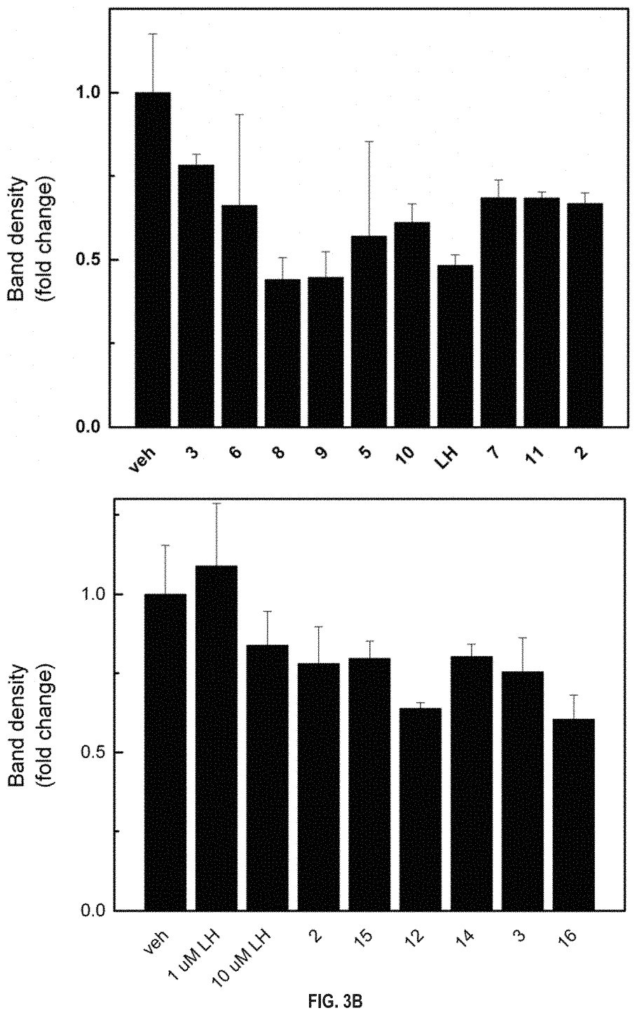

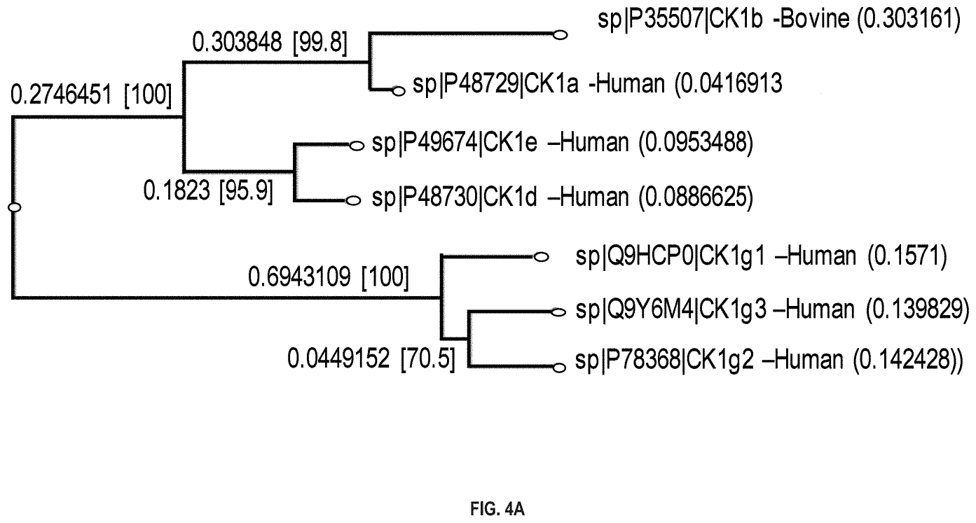

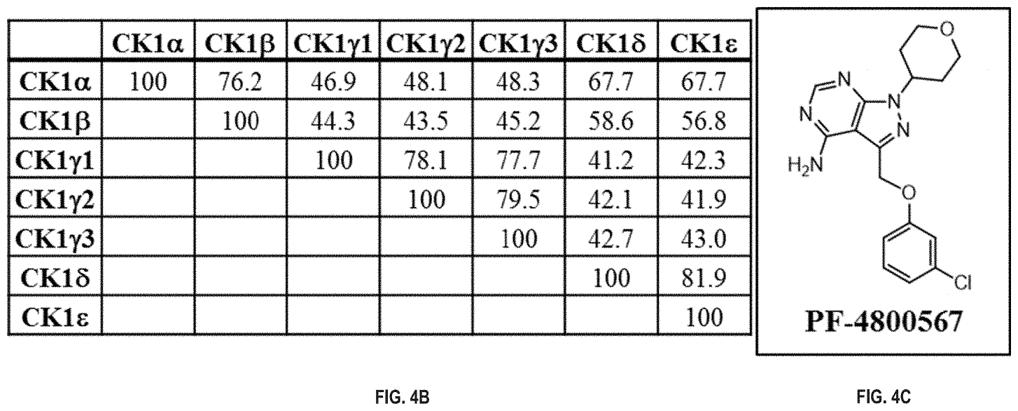

Inhibition of Tau Phosphorylation. To confirm biological relevance, the compounds identified by the FRET screens as potential CK1δ inhibitors were tested in an in vitro assay for their ability to inhibit the phosphorylation of tau at serine 202, a known target of CK1δ. LH 846, a known inhibitor of CK1δ was used as the positive control. The results are set forth in FIG. 3. Our results show that compound 5, 8, 9, 12 and 16 (from FIG. 1) exhibited comparable inhibition potency for the inhibition of tau phosphorylation at Ser202. In in-vitro tau phosphorylation assay in HeLa cells that were transfected with full length tau, the CK1δ inhibitors indeed lowered the tau phosphorylation levels at Ser202 by 20%-56%. Compounds 8 and 9 inhibited tau phosphorylation of Ser202 by 56% and 55%, respectively when added to HeLa cells that were transfected with and therefore overexpressed tan protein (FIG. 3). Compound 5 caused an inhibition of approximately 43%. These results, in combination with the FRET binding data suggest that compounds 5, 8, 9, 12 and 16 (from FIG. 1) may have the highest potential as a therapeutic for AD.

While the tau phosphorylation inhibition percentage was comparable for compounds 5, 8, 9, 12 and 16, compound 2 did not measure up to them in the inhibition of tau phosphorylation. One thought is that the ester linkage might have cleaved during the assay and the resulting 5,8-dihydroxy-1,4-naphthoquinone was not a good competitive inhibitor of CK1δ.

Thus, the search for new molecules that can inhibit CK1δ and subsequently also inhibit tau phosphorylation has yielded a series of hydroxynaphthoquinone and hydroxyanthraquinone analogs. These compounds inhibited CK1δ with low micromolar and submicromolar inhibition potency. Several of these CK1δ inhibitors were also effective in inhibiting tau phosphorylation at the residue Ser202. This example proves that CK1δ inhibitors can effectively function as potential therapeutic agents for AD in that these CK1δ inhibitors can inhibit the phosphorylation of tau protein at the critical residues involved in its interaction with microtubules. These CK1δ inhibitors may also reduce the tau phosphorylation at other residues that are known to be phosphorylated by CK1δ, as several of these additional residues on tau are known to play a role in microtubule stabilization. The docking studies (FIG. 2) have clearly indicated that a single carbonyl at the appropriate position would be sufficient to retain the activity of the compounds. This will eliminate the requirement of a quinone moiety. Similarly, 1,4-dihydroxybenzene ring fused to the quinone is redundant. New bicyclic and tricyclic molecules that incorporate the carbonyl that can hydrogen bond with Tyr56, an aromatic ring that can have n-methyl interactions with the lie residues in the binding pocket and a hydroxyl group that can hydrogen bond to the hinge region residues will enhance the potency of these molecules.

### Example 2—CK1ε Inhibitors

Selective inhibitors of CK1ε and/or CK1δ can function as in-vivo tools to decipher the distinct roles of these isozymes in cancer and in neurodegenerative disorders. To date only one molecule, PF-4800567, has been reported to show selectivity for CK1ε over CK1δ [40]. The X-ray crystal structure of PF-4800567 with CK1ε indicated that the selectivity is due to a flipped DFG motif and the resultant interaction of Phe150 with the chlorophenyl group of PF-4800567

The question then arises as to whether specific structural features can contribute to such selectivity for CK1ε. This example is directed to the identification of inhibitor structural features that contribute to the selective inhibition of CK1ε over CK1δ. Docking studies indicate that a similar flipped DFG motif and an interaction with Phe150 could be in play for such structural motifs.

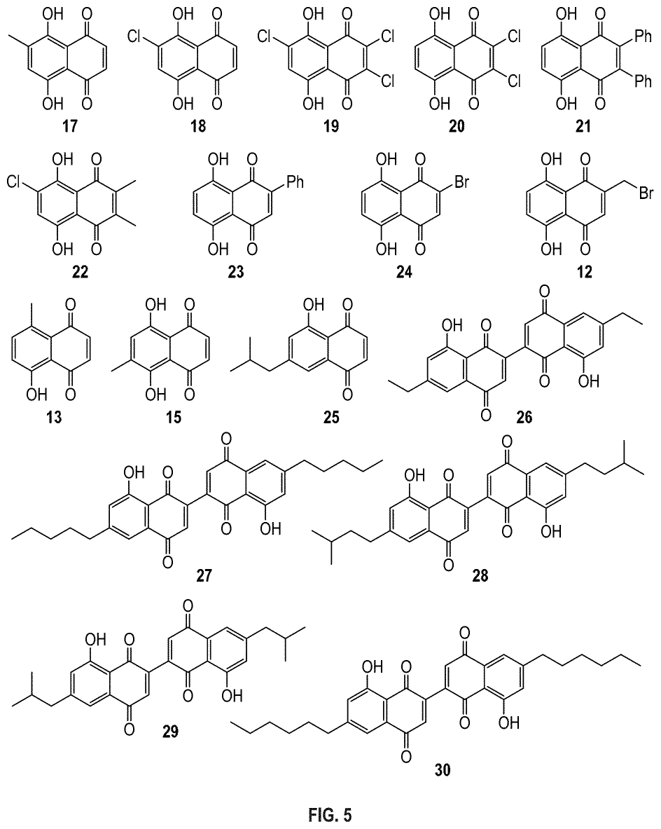

5-Hydroxy and 5,8-dihydroxy 1,4-anthraquinones were developed and synthesized as CK1ε inhibitors, and their structures are set forth in FIG. 5. Friedel-Crafts acylation reaction and Diels-Alder [4+2] cycloaddition reaction were used to synthesize the compounds depicted in FIG. 5.

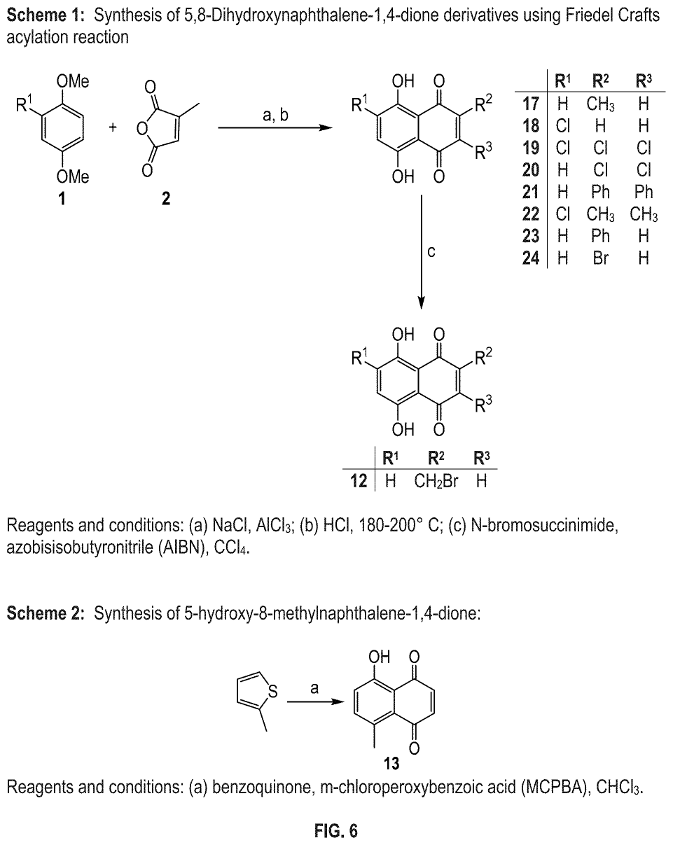

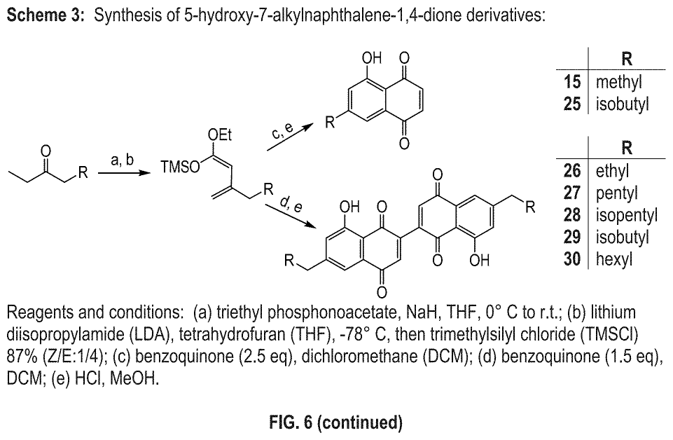

FIG. 6 sets forth the synthesis of the compounds from FIG. 5. The first scheme involves the Friedel-Crafts acylation reaction between a substituted maleic anhydride and a substituted 1,4-dimethoxybenzene in the presence of aluminum chloride followed by demethylation of the phenolic methyl ethers with IN hydrochloric acid in methanol to yield substituted 5,8-dihydroxynaphthalene-1,4-dione compounds 17 to 24. The side chain methyl group of compound 17 was then subjected to free radical bromination to obtain the compound 12. Diels-Alder [4+2] cycloaddition reaction of 2-methylthiophene and 1,4-benzoquinone, with m-chloroperoxybenzoic acid in chloroform for 48 h followed by silica gel chromatography was used for synthesizing compound 13 as illustrated in scheme 2. For the Diels-Alder reaction shown in scheme 3, the diene was initially made in two steps starting with the Wittig reaction of ketone of interest with ethyl (triphenylphosphoranylidene)acetate in DCM at room temperature to form the α,β-unsaturated ester that is then treated with lithium diisopropylamide followed by trimethylsilyl chloride in the second step to form the 3 and/or 4-substituted dienes 1-ethoxy-1-trimethylsiloxy-1,4-diene. This diene was then reacted with 1,4-benzoquinone, the dienone to form the monomer or dimer 5-hydroxynaphthalene-1,4-dione series of products (compounds 15, and 25 to 30) Formation of the monomer or dimer was dictated by the stoichiometry of the 1,4-benzoquinone used in the reaction. Use of 1.5 equivalents of 1,4-benzoquinone with respect to one equivalent of the diene resulted in the formation of the dimers 26 to 30. A large excess of 1,4-benzoquinone (2.5 equivalents) under similar reaction conditions yielded the monomer 15 and 25.

The synthesized molecules from FIG. 5 were then analyzed for the inhibition of CK1δ and CK1ε, using in-vitro kinase inhibition assay. The in-vitro kinase inhibition assays were conducted at the ThermoPisher Select Screen Biochemical Kinase Profiling Service. It is a Z′-LYTE biochemical FRET based fluorescence assay with a coupled enzyme format that has differential sensitivity to phosphorylated and non-phosphorylated peptides upon proteolytic cleavage. An initial high-throughput in-vitro screening assay at a concentration of 10 μM for all of the compounds in FIG. 5. The results are set forth in Table 3.

TABLE 3
Percentage inhibition of CK1δ and CK1ε by the
studied compounds at 10 μM concentration

| | % Inhibition | % Inhibition |
|---|---|---|
| Compound | CK1δ | CK1ε |
| 18 | 13 | 90 |
| 19 | 42 | 88 |
| 20 | 21 | 84 |
| 21 | 8 | 14 |
| 22 | 2 | 39 |
| 23 | 17 | 60 |
| 24 | 1 | 96 |
| 12 | 94 | 101 |
| 13 | 85 | 98 |
| 15 | 31 | 58 |
| 25 | 11 | 48 |
| 26 | 43 | 64 |
| 27 | 9 | 13 |
| 28 | 9 | 13 |
| 29 | 8 | 15 |
| 30 | 6 | 12 |

The results (table 3) indicated that compounds 18, 20, and 24 (from FIG. 5) showed >80% inhibition of CK1ε with >4 fold selectivity for CK1ε over CK1δ. Compounds 12 and 13 exhibited equal inhibition potency (>80%) for both CK1δ and CK1ε. Compound 19 showed 99% inhibition of CK1ε with a 2-fold selectivity for CK1δ over CK1δ. All other compounds showed weak to moderate inhibition for CK1δ/CK1ε with some of them indicating selectivity for CK1ε cover CK1δ (2-4 fold).

Compounds that presented >80% inhibition for CK1δ and/or CK1ε were then subjected to 10-point titrations, with a 3-fold dilution starting from the compound concentration of 25 μM to generate the dose-response curves (table 4). The compounds that showed selectivity for CK1ε in the high-throughput screening indicated a similar trend in the dose-response curves. Table 4 sets forth the IC₅₀ values of inhibition of CK1δ and CK1ε by some of the compounds from FIG. 5.

TABLE 4
IC₅₀ values of inhibition of CK1δ and CKlε
by the compounds 12, 13, 18, 19, 20 and 24.

| Compound | CK1δ | CK1ε |
|---|---|---|
| 18 | >25 | 1.34 |
| 19 | 9.58 | 3.98 |
| 20 | >25 | 7.52 |
| 24 | >25 | 2.10 |
| 12 | 0.72 | 0.19 |
| 13 | 2.41 | 1.19 |

The IC₅₀ values for compounds 18, 20, and 24 (from FIG. 5) for CK1ε were 1.34 μM, 7.52 μM and 2.10 μM, respectively. The IC₅₀ values for these compounds (18, 20, and 24) for CK1δ could not be determined as they did not inhibit the kinase significantly at the highest concentration of 25 μM. Compounds 19, 12 and 13 inhibited both kinases in almost equal measure in the high-throughput screening. The dose-response curve results demonstrated a similar trend in inhibition of both kinases to a similar extent. The IC₅₀ values for compounds 19, 12 and 13 for CK1δ were 9.58 μM, 0.72 μM and 2.41 μM, respectively and for CK1ε were 1.34 μM, +1.19 μM and 1.19 μM, respectively. The kinase inhibition data clearly indicate that the compounds 18, 20, and 24 (from FIG. 5) contained structural features that imparted selectivity in the inhibition of CK1ε over CK1δ.

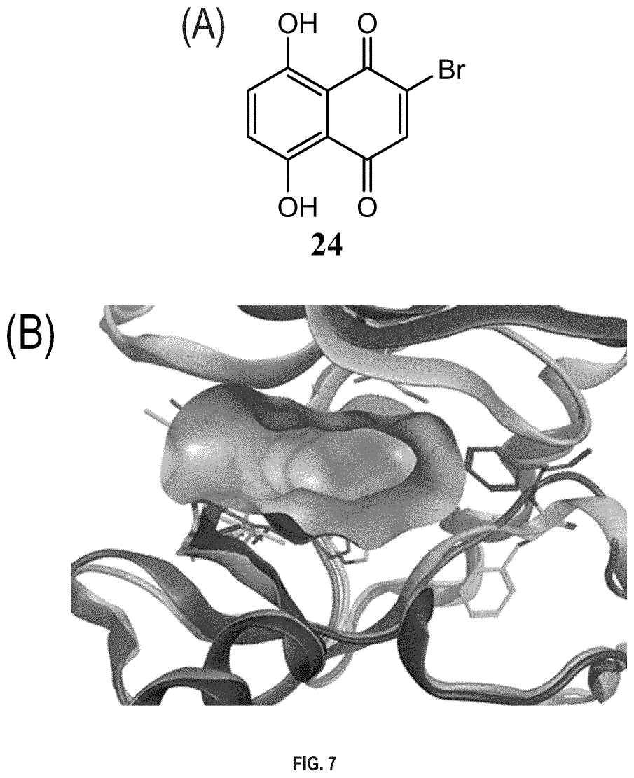

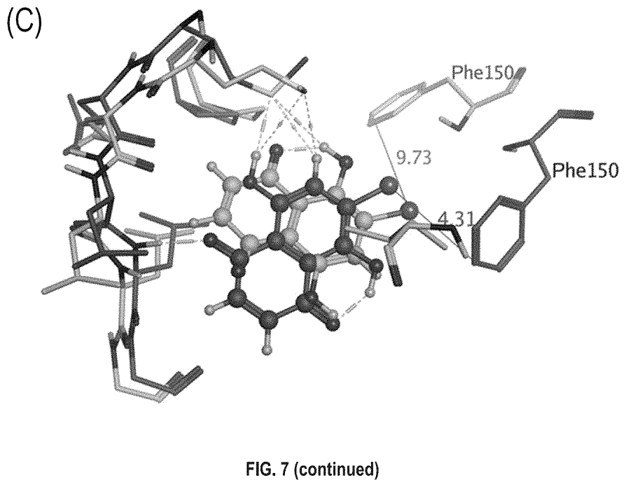

To understand the binding modes of these molecules and the nature of interactions with the residues in the binding pocket, docking studies were performed computationally using the MOE (Molecular Operating Environment) software from the ChemComp group. The crystal structures of CK1δ (4HGT.pdb), apo CK1ε (4HOK.pdb) and CK1ε (4HNI.pdb) in complex with PF-4800567 were taken from the Protein Data Bank website. X-Ray crystal studies reported by Long et al on the CK1ε bound to PF-4800567 showed that the binding of the CK1ε selective inhibitor PF-4800567 to CK1ε resulted in a flip in the DFG motif that enabled the Phe150 residue to have interactions with the chlorophenyl ring of PF-4804567. The apo CK1ε structure has Phe150 of the DFG motif adopts a DFG-in conformation that has the residue buried in a hydrophobic pocket comprised of Tyr56, Met59, Ile15, Ile119, His126 and Ile147. When the DFG-out conformation is evidenced with the 180° flip of this motif, the Phe150 swings out of this hydrophobic pocket resulting in the aromatic side chain residing at an ideal distance for it to have favorable interactions with the bound ligand. The void created by the swing-out of Phe150 from the hydrophobic pocket is filled by the residue Phe55 of the C-α helix that is rotated. Such a conformational change is possible in CK1ε but not in CK1δ as the residue 55 in CK1δ is lie that is not capable of interacting in a similar manner. To check whether the CK1ε selective compounds show interactions with Phe150 in the DFG-out conformation, the compounds 18, 20, and 24 docked onto the CK1ε 3D-structure which has the DFG-out motif (FIG. 7). The compounds did indeed show interaction of the side chain halogens with the Phe150 aromatic ring when the DFG motif was flipped. FIG. 8 shows the binding modes of compound 24 with the CK1ε and CK1δ active site residues. The distance between the side-chain bromine atom and the Phe150 residue is 11.27 A in the CK1δ bound complex. When the same molecule binds to the DFG-out conformational structure of CK1ε, the side-chain bromine atom is at a distance of 3.85 A from with the phenyl side chain of Phe150 indicating an aromatic-halogen interaction. This example has identified structural features that can impart selectivity for the inhibition of CK1ε over CK1δ.

All references cited in this specification are herein incorporated by reference as though each reference was specifically and individually indicated to be incorporated by reference. The citation of any reference is for its disclosure prior to the filing date and should not be construed as an admission that the present disclosure is not entitled to antedate such reference by virtue of prior invention.

It will be understood that each of the elements described above, or two or more together may also find a useful application in other types of methods differing from the type described above. Without further analysis, the foregoing will so fully reveal the gist of the present disclosure that others can, by applying current knowledge, readily adapt it for various applications without omitting features that, from the standpoint of prior art, fairly constitute essential characteristics of the generic or specific aspects of this disclosure set forth in the appended claims. The foregoing embodiments are presented by way of example only; the scope of the present disclosure is to be limited only by the following claims.

### REFERENCES CITED

1. Association. A. s., Alzheimer's Disease Facts and Figures. Alzheimers Dement 2019, 15 (3), 321-387.
2. Alzheimer's Disease International: World Alzheimer Report. 2018.
3. Bertram, L.; Lill, C. M.; Tanzi, R. E., The genetics of Alzheimer disease: back to the future. Neuron 2010, 68 (2), 270-81.
4. Citron, M., Alzheimer's disease: strategies for disease modification. Nat Rev Drug Discov 2010, 9 (5), 387-98.
5. Cohen, T. J.; Guo, J. L.; Hurtado, D. E.; Kwong, L. K.; Mills, I. P.; Trojanowski, J. Q.; Lee, V. M., The acetylation of tau inhibits its function and promotes pathological tau aggregation. Nat Commun 2011, 2, 252
6. De Strooper, B.; Vassar, R.; Golde, T., The secretases: enzymes with therapeutic potential in Alzheimer disease. Nat Rev. Neurol 2010, 6 (2), 99-107.
7. Iqbal, K.; Liu, F.; Gong, C. X.; Grundke-Iqbal, I., Tau in Alzheimer disease and related tauopathies. Curr Alzheimer Res 2010, 7(8). 656-64.
8. Min, S. W.; Cho, S. H.; Zhou, Y.; Schroeder, S.; Haroutunian, V.; Seeley, W. W.; Huang, E. J.; Shen, Y.; Masliah, E.; Mukherjee, C.; Meyers, D.; Cole, P. A.; Ott, M.; Gan, Acetylation of tau inhibits its degradation and contributes to tauopathy. Neuron 2010, 67 (6), 953-66.
9. Morns, M.; Maeda, S.; Vossel, K.; Mucke, L.; The many faces of tau. Neuron 2011, 70 (3), 410-26.
10. Prasher, V. P.; Farrer, M. J.; Kessling, A. M.; Fisher, E. M.; West, R. J.; Barber, P. C.; Butler, A. C., Molecular mapping of Alzheimer-type dementia in Down's syndrome. Ann Neurol 1998, 43 (3). 380-3.
11. Huang, Y.; Mucke, L., Alzheimer mechanisms and therapeutic strategies. Cell 2012, 148 (6), 1204-22.
12. Sanabria-Castro, A; Alvarado-Echeverria, I.; Monge-Bonilla, C., Molecular Pathogenesis of Alzheimer's Disease: An Update. Ann Neurosci 2017, 24 (l), 46-54.
13. Koh, M. T.; Haberman, R. P.; Foti, S.; McCown. T. J.; Gallagher, M., Treatment strategies targeting excess hippocampal activity benefit aged rats with cognitive impairment. Neuropsychopharmacology 2010, 35 (4), 1016-25.
14. Palop, J. J.; Mucke, L., Amyloid-beta-induced neuronal dysfunction in Alzheimer's disease: from synapses toward neural networks. Nat Neurosci 2010, 13 (7), 812-8.
15. Cohen, E.; Paulsson, J. F.; Blinder, P.; Burstyn-Cohen, T.; Du. D.; Estepa, G.; Adame, A.; Pham, H. M.; Holzenberger, M.; Kelly, J. W.; Masliah. E.; Dillin. A., Reduced IGF-1 signaling delays age-associated proteotoxicity in mice. Cell 2009, 139(6), 1157-69.
16. Gan. L.; Mucke, L., Paths of convergence: sirtuins in aging and neurodegeneration Neuron 2008, 58 (1). 10-4.
17. Harris, H.; Rubinsztein, D. C., Control of autophagy as a therapy for neurodegenerative disease. Nat Rev Neurol 2011, 8 (2), 108-17.
18. Kopke, E.; Tung, Y. C.; Shaikh, S.; Alonso, A. C.; Iqbal, K.; Grundke-Iqbal, I., Microtubule-associated protein tau. Abnormal phosphorylation of a non-paired helical filament pool in Alzheimer disease. J Biol Chem 1993, 268 (32), 24374-84.
19. Alonso, A. D.; Grundke-Iqbal, I.; Barra. H. S.; Iqbal, K., Abnormal phosphorylation of tau and the mechanism of Alzheimer neurofibrillary degeneration: sequestration of microtubule-associated proteins 1 and 2 and the disassembly of microtubules by the abnormal tau. Proc Natl Acad Sci USA 1997, 94 (1), 298-303.
20. Alonso, A. C.; Zaidi, T.; Grundke-Iqbal, L; Iqbal, K., Role of abnormally phosphorylated tau in the breakdown of microtubules in Alzheimer disease. Proc Natl Acad Sci USA 1994, 91 (12), 5562-6.
21. Wang, J. Z.; Gong. C. X.; Zaidi, T.; Gundke-Iqbal, I; Iqbal, K., Dephosphorylation of Alzheimer paired helical filaments by protein phosphatase-2A and -2B. J Biol Chem 1995, 270 (9), 4854-60.
22. Wang, J. Z.; Grundke-Iqbal, I.; Iqbal, K., Restoration of biological activity of Alzheimer abnormally phosphorylated tau by dephosphorylation with protein phosphatase-2A, -2B and -1. Brain Res Mol Brain Res 1996, 38 (2). 200-8.
23. Wang, J. Z.; Wu. Q.; Smith. A.; Grundke-Iqbal, I.; Iqbal, K., Tau is phosphorylated by GSK-3 at several sites found in Alzheimer disease and its biological activity markedly inhibited only after it is pyrophosphorylated by A-kinase. FEBS Lett 1998, 436 (1), 28-34.
24. Knippschild, U.; Gocht, A.; Wolff, S.; Huber. N.; Lohler, J.; Stoter, M., The casein kinase 1 family: participation in multiple cellular processes in eukaryotes. Cell Signal 2005, 17 (6), 675-89.
25. Elmore, Z. C.; Guillen, R. X.; Gould, K. L., The kinase domain of CK1 enzymes contains the localization cue essential for compartmentalized signaling at the spindle pole. Mol Biol Cell 2018, 29 (13), 1664-1674
26. Ghalei, H.; Schaub, F. X.; Doherty, I. R.; Noguchi, Y.; Roush, W. R.; Cleveland, J. L.; Stroupe, M. E.; Karbstein, K., Hrr25/CK1delta-directed release of Ltv1 from pre-40S ribosomes is necessary for ribosome assembly and cell growth. J Cell Biol 2015, 208 (6), 745-59.
27. Knippschld, U.; Wolff. S.; Giamas, G.; Brockschmidt, C.; Wittau, M.; Wurl, P. U.; Eismann, T.; Stoter, M., The role of the casein kinase 1 (CK1) family in different signaling pathways linked to cancer development. Onkologie 2005, 28(10), 508-14.
28. Nakatogawa, H., Regulated degradation: controlling the stability of autophagy gene transcripts. Dev Cell 2015, 34 (2), 132-4.
29. Schittek, B., Sinnberg, T., Biological functions of casein kinase 1 isoforms and putative roles in tumorigenesis. Mol Cancer 2014, 13, 231.
30. Gross, S. D.; Anderson, R. A., Casein kinase 1: spatial organization and positioning of a multifunctional protein kinase family. Cell Signal 1998, 10(10), 699-711.
31. Price, M. A., CK1, there's more than one: casein kinase 1 family members in Wnt and Hedgehog signaling. Genes Dev 2006, 20 (4), 399-410.
32. Rubinfeld, B.; Tice, D. A.; Polakis, P., Axin-dependent phosphorylation of the adenomatous polyposis coli protein mediated by casein kinase 1epsilon. J Biol Chem 2001, 276 (42), 39037-45.
33. Rena, G.; Bain, J.; Elliott, M.; Cohen, P, D4476, a cell-permeant inhibitor of CK1, suppresses the site-specific phosphorylation and nuclear exclusion of FOXO1a. EMBO Rep 2004, 5 (1), 60-S.
34. Shen, T.; Liu, Y., Cseresnyes, Z.; Hawkins, A.; Randall, W. R.; Schneider, M. F., Activity- and calcineurin-independent nuclear shuttling of NFATc1, but not NFATc3, in adult skeletal muscle fibers. Mol Biol Cell 2006, 17(4), 1570-82.
35. Wu, S.; Chen, L.; Becker, A.; Schonbrunn, E.; Chen, J., Casein kinase 1alpha regulates an MDMX intramolecular interaction to stimulate p53 binding. Mol Cell Biol 2012, 32 (23), 4821-32.
36. Alappat, E. C.; Feig, C.; Boyerinas, B.; Volkland, J.; Samuels, M.; Murmann, A. E.; Thorburn, A.; Kidd, V. J.; Slaughter, C. A.; Osborn, S. L.; Winoto, A.; Tang, W. J.; Peter, M. E., Phosphorylation of FADD at serine 194 by CK1alpha regulates its nonapoptotic activities. Mol Cell 2005, 19(3), 321-32.
37. Harms, E.; Young, M. W.; Saez, L., CK1 and GSK3 in the Drosophila and mammalian circadian clock. Novartis Found Sym 2003, 253, 267-77; discussion 102-9, 277-84.
38. Hirota, T.; Lee, J. W.; Lewis, W. G.; Zhang, E. E.; Breton, G.; Liu. X.; Garcia, M.; Peters, E. C.; Etchegaray, J. P.; Traver, D.; Schultz, P. G.; Kay, S. A., High-throughput chemical screen identifies a novel potent modulator of cellular circadian rhythms and reveals CKIalpha as a clock regulatory kinase. PLoS Biol 2010, 8 (12), e1000559.
39. Meng, Q. J., Maywood, E. S.; Bechtold, D. A.; Lu. W. Q.; Li. J.; Gibbs, J. E.; Dupre, S. M.; Chesham, J. E.; Rajamohan, F.; Knafels, J.; Sneed, B.; Zawadzke, L. E.; Ohren, J. F.; Walton, K. M.; Wager, T. T.; Hastings, M. H.; Loudon, A. S., Entrainment of disrupted circadian behavior through inhibition of casein kinase 1 (CK1) enzymes. Proc. Natl Acad Sci USA 2010, 107 (34), 15240-5.
40. Walton, K. M.; Fisher, K.; Rubitski, D.; Marconi, M.; Meng, Q. J.; Sladek. M.; Adams, J.; Bass, M.; Chandrasekaran, R.; Butler, T.; Griffor, M.; Rajamohan, F.; Serpa, M.; Chen, Y.; Claffey, M.; Hastings, M.; Loudon, A.; Maywood, E.; Ohren, J.; Doran, A.; Wager, T. T., Selective inhibition of casein kinase 1 epsilon minimally alters circadian clock period. J Pharmacol Exp Ther 2009, 330 (2), 430-9
41. Greer, Y. E.; Westlake, C. J.; Gao, B.; Bharti, K.; Shiba, Y.; Xavier, C. P.; Pazour, G. J.; Yang, Y.; Rubin, J. S., Casein kinase 1 delta functions at the centrosome and Golgi to promote ciliogenesis. Mol Biol Cell 2014, 25 (10), 1629-40.
42. Hanger, D. P.; Byers, H. L.; Wray, S.; Leung, K. Y.; Saxton, M. J.; Seereeram, A.; Reynolds, C. H., Ward, M. A; Anderton, B. H., Novel phosphorylation sites in tau from Alzheimer brain support a role for casein kinase 1 in disease pathogenesis. J Biol Chem 2007, 282 (32). 23645-54.
43. Leon-Espinosa, G.; Garcia, E.; Garcia-Escudero, V.; Hernandez, F.; Defelipe, J.; Avila. J., Changes in tau phosphorylation in hibernating rodents. J Neurosci Res 2013, 91 (7), 954-62.
44. Li. G.; Yin, H.; Kuret, J., Casein kinase 1 delta phosphorylates tau and disrupts its binding to microtubules. J Biol Chem 2004, 279(16). 15938-45.
45. Stoter, M.; Bamberger. A. M.; Asian, B.; Kurth, M.; Speidel, D.; Loning, T.; Frank, H. G., Kaufmann, P.; Lohler, J.; Henne-Bruns, D.; Deppert, W.; Knippschild, U., Inhibition of casein kinase 1 delta alters mitotic spindle formation and induces apoptosis in trophoblast cells. Oncogene 2005, 24 (54). 7964-75.
46. Chen, C.; Gu, J.; Basurto-Islas. G.; Jin, N.; Wu. F.; Gong, C. X.; Iqbal, K.; Liu, F., Up-regulation of casein kinase 1 epsilon is involved in tau pathogenesis in Alzheimer's disease. Sci Rep 2017, 7(1), 13478.
47. Hanger, D. P.; Byers, H. L.; Wray, S.; Leung, K. Y.; Saxton, M. J.; Seereeram, A.; Reynolds, C. H.; Ward, M. A; Anderton, B. H., Novel phosphorylation sites in tau from Alzheimer brain support a role for casein kinase 1 in disease pathogenesis. J Biol Chem 2007, 282 (32), 23645-54.
48. Li, G.; Yin, I.; Kuret, J., Casein kinase 1 delta phosphorylates tau and disrupts its binding to microtubules. J Biol Chem 2004, 279 (16), 15938-45.
49. Ghoshal, N.; Smiley, J. F.; DeMaggio, A. J.; Hoekstra, M. F.; Cochran, E. J.; Binder, L. T.; Kuret., J., A new molecular link between the fibrillar and granulovacuolar lesions of Alzheimer's disease. Am J Pathol 1999, 155 (4), 1163-72.
50. Kuret, J., Johnson, G. S.; Cha, D.; Christenson. E. R.; DeMaggio, A. J.; Hoekstra, M. F., Casein kinase 1 is tightly associated with paired-helical filaments isolated from Alzheimer's disease brain. J Neurochem 1997, 69 (6), 2506-15.
51. Wolff, S.; Xiao, Z.; Wittau, M.; Sussner, N.; Stoter, M.; Knippschild, U., Interaction of casein kinase 1 delta (CK1 delta) with the light chain LC2 of microtubule associated protein 1A (MAP1A). Biochim Biophys Acta 2005, 1745 (2), 196-206.
52. Wager, T. T.; Chandrasekaran, R. Y.; Bradley, J.; Rubitski, D.; Berke, H.; Mente, S.; Butler, T.; Doran. A.; Chang. C.; Fisher, K.; Knafels. J.; Liu, S.; Ohren, J.; Marconi, M.; DeMarco, G.; Sneed. B.; Walton, K.; Horton, D.; Rosado, A.; Mead, A., Casein kinase 1delta/epsilon inhibitor PF-5006739 attenuates opioid drug-seeking behavior. ACS Chem Neurosci 2014, 5 (12), 1253-65.
53. Peifer, C.; Abadleh, M.; Bischof, J.; Hauser, D.; Schattel, V.; Hirner, H.; Knippschild, U.; Laufer, S., 3,4-Diaryl-isoxazoles and -imidazoles as potent dual inhibitors of p38alpha mitogen activated protein kinase and casein kinase 1delta. J Med Chem 2009, 52 (23), 7618-30.
54. Hua, Z.; Huang, X.; Bregman, H.; Chakka, N.; DiMauro, E. F.; Doherty, E. M.; Goldstein, J.; Gunaydin, H.; Huang, H.; Mercede, S.; Newcomb. J.; Patel, V. F.; Turci, S. M.; Yan, J.; Wilson. C.; Martin, M. W., 2-Phenylamino-6-cyano-1H-benzimidazole-based isoform selective casein kinase 1 gamma (CK1gamma) inhibitors. Bioorg Med Chem Left 2012, 22 (17), 5392-5.
55. Sciabola, S.; Benedetti, P.; D'Arrigo. G.; Torella, R.; Baroni, M.; Cruciani, G.; Spyrakis, F., Discovering New Casein Kinase 1d Inhibitors with an Innovative Molecular Dynamics Enabled Virtual Screening Workflow. ACS Med Chem Lett 2019, 10 (4), 487-492.
56. Behrend, L.; Milne, D. M.; Stöter, M.; Deppert, W.; Campbell, L. E.; Meek, D. W.; Knippschild, U., IC261, a specific inhibitor of the protein kinases casein kinase 1-delta and -epsilon, triggers the mitotic checkpoint and induces p53-dependent postmitotic effects. Oncogene 2000, 19 (47), 5303-5313.
57. Cozza, G.; Gianoncelli, A.; Montopoli, M.; Caparrotta, L.; Venerando, A.; Meggio, F.; Pinna, L. A.; Zagotto, G.; Moro, S., Identification of novel protein kinase CK1 delta (CK1delta) inhibitors through structure-based virtual screening. Bioorg Med Chem Lett 2008, 18 (20), 5672-5.
58. Long, A. M.; Zhao, H.; Huang, X., Structural basis for the potent and selective inhibition of casein kinase 1 epsilon. J Med Chem 2012, 55 (22), 10307-11.

1. A method of treating a serine/threonine kinase dependent disease comprising administering to a subject a compound according to Formula (II): 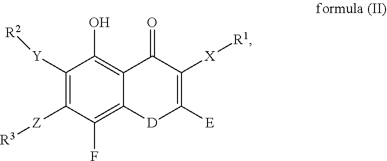
wherein:
X, Y and Z independently represent a direct bond, —C(R⁴)—, —O—, —S—, —OH, —NH₂, —CH₂O—, CH₂S—, —(CH₂)₂O—, —NR⁵—, —NR⁵CH₂—, —CH₂NR⁵—, —NR⁵CO—, —CONR⁵—, —N═N—, —NH—CO—NH—, —NH—CS—NH—, —CO—O—, CO—O—CH₂—, —SO₂NH—, —NH—SO₂—, —CR⁴═CR⁴—, —C≡C—, —O—CH₂—CO—, —OCH₂CH₂O—, —CH(OH)—, —NO₂ bridging groups,
R⁴ represents hydrogen, C₁₋₆ alkyl, C₁₋₆ alkenyl, C₁₋₆ alkoxy, C₁₋₆ haloalkyl, haloC₁₋₆ alkoxy, —OH, —(═O), —COOH, —CONH₂, —COC₁₋₆ alkyl, O—C₁₋₆ alkyl or alkenyl or alkynyl, NH—C₁₋₆ alkyl or alkenyl or alkynyl, —SC₁₋₆ alkyl groups, —(═S), CSSH, CSNH₂, —CSC₁₋₆ alkyl or alkenyl or alkynyl, S—C₁₋₆ alkyl or alkenyl or alkynyl
R¹, R², R³ and R⁵ independently represent hydrogen, C₁₋₆ alkyl, alkenyl, alkynyl, halogenated or hydroxyl alkyl, alkenyl, alkynyl, halogenated or hydroxyl or amino-alkenyl, halogenated or hydroxyl or amino-alkynyl, halogen, aryl, C₃₋₈ cycloalkyl, monocyclic or bicyclic heterocyclyl, monocyclic or bicyclic heteroaryl, wherein the aryl, heteroaryl or heterocyclyl groups may be optionally substituted by one or more R⁴ groups,
D represents —(C═O)—, —(CH₂)ₙ— where n=0, 1, 2, —CHOH—, CHNH₂—, —O—, —S—, —NH—, —N—CH₃—,
E represents hydrogen, C₁₋₆ alkyl, halogen, —OH, aryl, halogenated/hydroxyl aryl, heteroaryl, halogenated or hydroxyl or amino-heterocyclyl, cycloalkyl, halogenated or hydroxyl alkyl, alkenyl, alkynyl, halogenated or hydroxyl or amino-alkenyl, halogenated or hydroxyl or amino-alkynyl,
F represents hydrogen, C₁₋₆ alkyl, —OH, —NH2, NHCOCH₃, NHCOR¹, aryl, halogenated/hydroxyl aryl, heteroaryl, halogenated or hydroxyl or amino-heterocyclyl, cycloalkyl, halogenated or hydroxyl alkyl, alkenyl, alkynyl, halogenated or hydroxyl or amino-alkenyl, halogenated or hydroxyl or amino-alkynyl;
and/or a pharmaceutically acceptable salt, and/or solvate thereof.

2. The method according to claim 1, wherein:
X, Y and Z independently represent a direct bond, —C(R⁴)—, or —CH(OH)—,
R⁴ represents hydrogen, C₁₋₆ alkyl, halogen, —(═O), or —OH,
R¹, R², and R³ independently represent hydrogen, C₁₋₆ alkyl, halogen, aryl, C₃₋₈ cycloalkyl, monocyclic or bicyclic heterocyclyl, monocyclic or bicyclic heteroaryl, wherein the aryl, heteroaryl or heterocyclyl groups may be optionally substituted by one or more R⁴ groups,
D represents —(C═O)—,
E represents aryl, hydrogen, C₁₋₆ alkyl, or halogen,
F represents hydrogen, C₁₋₆ alkyl, or —OH.

3. The method according to claim 1, wherein:
D is —(C═O)—, and F is —OH or H.

4. The method according to claim 1, wherein:
D is —(C═O)—, and F is —OH.

5. The method according to claim 1, wherein:
X, Y and Z independently represent a direct bond, —C(R⁴)—, or —CH(OH)—,
R⁴ represents halogen,
R¹, R², and R³ independently represents hydrogen, halogen, or C₁₋₆ alkyl,
D represents —(C═O)—,
E represents hydrogen, and
F represents —OH, and
the compound according to Formula (II) is an inhibitor of CK1δ.

6. The method according to claim 1, wherein:
X, Y and Z independently represent a direct bond,
R¹, R², and R³ independently represent hydrogen or halogen,
D represents —(C═O)—,
E represents hydrogen or halogen,
F represents —OH, and
the compound is an inhibitor of CK1ε.

7. A method of treating a neurodegenerative disease comprising administering to a subject in need of such treatment a compound according to Formula (II): 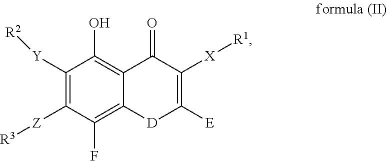
wherein:
X, Y and Z independently represent a direct bond, —C(R⁴)—, —O—, —S—, —OH, —NH₂, —CH₂O—, CH₂S—, —(CH₂)₂O—, —NR⁵—, —NR⁵CH₂—, —CH₂NR⁵—, —NR⁵CO—, —CONR⁵—, —N═N—, —NH—CO—NH—, —NH—CS—NH—, —CO—O—, CO—O—CH₂—, —SO₂NH—, —NH—SO₂—, —CR⁴═CR⁴—, —C≡C—, —O—CH₂—CO—, —OCH₂CH₂O—, —CH(OH)—, —NO₂ bridging groups,
R⁴ represents hydrogen, C₁₋₆ alkyl, C₁₋₆ alkenyl, C₁₋₆ alkoxy, C₁₋₆ haloalkyl, haloC₁₋₆ alkoxy, —OH, —(═O), —COOH, —CONH₂, —COC₁₋₆alkyl, O—C₁₋₆ alkyl or alkenyl or alkynyl, NH—C₁₋₆ alkyl or alkenyl or alkynyl, —SC₁₋₆ alkyl groups, —(═S), CSSH, CSNH₂, —CSC₁₋₆ alkyl or alkenyl or alkynyl, S—C₁₋₆ alkyl or alkenyl or alkynyl
R¹, R², R³ and R⁵ independently represent hydrogen, C₁₋₆ alkyl, alkenyl, alkynyl, halogenated or hydroxyl alkyl, alkenyl, alkynyl, halogenated or hydroxyl or amino-alkenyl, halogenated or hydroxyl or amino-alkynyl, halogen, aryl, C₃₋₈ cycloalkyl, monocyclic or bicyclic heterocyclyl, monocyclic or bicyclic heteroaryl, wherein the aryl, heteroaryl or heterocyclyl groups may be optionally substituted by one or more R⁴ groups,
D represents —(C═O)—, —(CH₂)ₙ— where n=0, 1, 2, —CHOH—, CHNH₂—, —O—, —S—, —NH—, —N—CH₃—,
E represents hydrogen, C₁₋₆ alkyl, halogen, —OH, aryl, halogenated/hydroxyl aryl, heteroaryl, halogenated or hydroxyl or amino-heterocyclyl, cycloalkyl, halogenated or hydroxyl alkyl, alkenyl, alkynyl, halogenated or hydroxyl or amino-alkenyl, halogenated or hydroxyl or amino-alkynyl,
F represents hydrogen, C₁₋₆ alkyl, —OH, —NH2, NHCOCH₃, NHCOR¹, aryl, halogenated/hydroxyl aryl, heteroaryl, halogenated or hydroxyl or amino-heterocyclyl, cycloalkyl, halogenated or hydroxyl alkyl, alkenyl, alkynyl, halogenated or hydroxyl or amino-alkenyl, halogenated or hydroxyl or amino-alkynyl;
and/or a pharmaceutically acceptable salt, and/or solvate thereof.

8. The method of claim 7, wherein said neurodegenerative disease is Alzheimer's disease.

9. A method of treating a serine/threonine kinase dependent disease comprising administering to a subject a compound of formula (III)-(X), a pharmaceutically acceptable salt, or solvate:

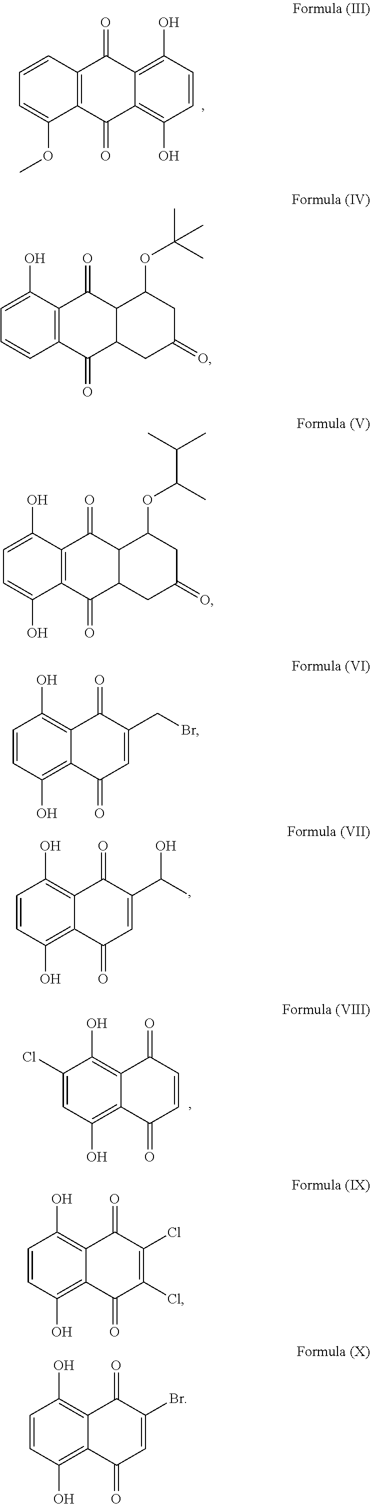

10. The method according to claim 9, wherein the serine/threonine kinase dependent disease is a neurodegenerative disease.

11. The method according to claim 9, wherein the serine/threonine kinase dependent disease is Alzheimer's disease.

12. The method according to claim 9, wherein the compounds of formula (III)-(VII) are CK1δ inhibitors.

13. The method according to claim 9, wherein the compounds of formula (VIII)-(X) are CK1ε inhibitors.

14. A compound according to Formula (II), and/or a stereoisomer and/or pharmaceutically acceptable salt and/or solvate thereof: 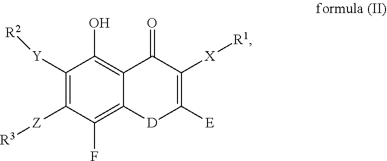
wherein:
X, Y and Z independently represent a direct bond or —C(R⁴)—
R⁴ represents hydrogen, C₁₋₆ alkyl,
R¹, R², and R³ independently represent hydrogen, C₁₋₆ alkyl, aryl, C₃₋₈ cycloalkyl, monocyclic or bicyclic heterocyclyl, monocyclic or bicyclic heteroaryl, wherein the aryl, heteroaryl or heterocyclyl groups may be optionally substituted by one or more R⁴ groups,
D represents —(C═O)—,
E represents hydrogen, halogen
F represents hydrogen, and
the compound is an inhibitor of CK1ε.

15. The compound of claim 14 for use in the treatment of a neurodegenerative disease in a mammal in need thereof.

16. The compound of claim 14 for use in the treatment of Alzheimer's disease in a mammal in need thereof.

17. The compound of claim 14 for use in inhibiting a serine/threonine kinase to treat a serine/threonine kinase-dependent disease in a mammal in need thereof.

18. The compound of claim 1, wherein the serine/threonine kinase to be inhibited is CK1δ and/or CK1ε.

19. A composition comprising the compound of claim 1 for use as a medicament.

20. A pharmaceutical composition comprising a compound, pharmaceutically acceptable salt, solvate, or composition of claim 1 and a pharmaceutically acceptable carrier.

21. The pharmaceutical composition of claim 20, suitable for enteral administration.

22. The pharmaceutical composition of claim 20, wherein said pharmaceutical composition is suitable for oral administration.

23. The pharmaceutical composition of claim 20, suitable for parenteral administration.
